(19) United States
(12) Patent Application Publication
(10) Pub. No.: US 2019/0117429 A1
(43) Pub. Date: Apr. 25, 2019

(54) REHABILITATION SHOE

(71) Applicant: YU-CHIEN WANG, Taichung City (TW)

(72) Inventor: YU-CHIEN WANG, Taichung City (TW)

(21) Appl. No.: 15/788,113

(22) Filed: Oct. 19, 2017

(51) Int. Cl. A61F 5/01

(52) U.S. Cl. CPC A61F 5/0127

(57) ABSTRACT

A rehabilitation shoe includes a shoe shell, a calf-supporting component, a shoe sole, a pedal and a shoe pad, the shoe shell has first engaging portions extending horizontally, the calf-supporting component is detachably arranged above a rear end of the shoe shell and extends upward, the shoe sole is made of a flexible material, an inner edge of a top portion of the shoe sole has second engaging portions extending horizontally, the second and first engaged portions are positionably engaged with each other so that the shoe sole is fixed on the bottom portion of the shoe shell; the pedal is made of a hard material and disposed on a top surface of the shoe shell; the shoe pad is made of a soft material and disposed on a top surface of the pedal, and the shoe pad is for a foot to step thereon.

### BACKGROUND OF THE INVENTION

### Field of the Invention

The present invention relates to a rehabilitation shoe.

### Description of the Prior Art

Conventionally, a rehabilitation shoe, as disclosed in TW480997, includes a sole part and a calf-supporting part, the sole part and the calf-supporting part are mostly integrally formed, and the sole part contacts a ground directly; therefore, a contact face is hard, and it is uncomfortable to wear the rehabilitation shoe. In addition, because the sole part and the calf-supporting part are mostly integrally formed, the movement of the ankle joint is restricted.

Furthermore, in the conventional rehabilitation shoe, the sole part is like a slipper, a toe part is hollowed-out. Although it is good for air permeability, the toes of a user often slide forward. Especially the rehabilitation shoe does not have various sizes like common shoes, there is a gap between the foot of the user and the rehabilitation shoe, if the toe part is hollowed-out, the foot of the user will slide back and forth when walking, and it will affect the comfort and safety of wearing the rehabilitation shoe.

Therefore, it is important to provide a rehabilitation shoe which has preferable support and structural strength and is comfortable and fit to wear.

### SUMMARY OF THE INVENTION

The major object of the present invention is to provide a rehabilitation shoe, which is easy to be manufactured and assembled, fit to a curve of a calf, and comfortable and solid.

To achieve the above and other objects, a rehabilitation shoe is provided, including a shoe shell, a calf-supporting component, a shoe sole, a pedal and a shoe pad.

The shoe shell has a plurality of first engaging portions which extend horizontally and are near a bottom portion of the shoe shell, the calf-supporting component is detachably arranged above a rear end of the shoe shell and extends upward, the shoe sole is made of a flexible material, an inner edge of a top portion of the shoe sole has a plurality of second engaging portions which extend horizontally, the second engaging portions and the first engaged portions are positionably engaged with each other so that the shoe sole is fixed on the bottom portion of the shoe shell; the pedal is made of a hard material and disposed on a top surface of the shoe shell; the shoe pad is made of a soft material and disposed on a top surface of the pedal, and the shoe pad is for a foot to step thereon.

A rehabilitation shoe of the present invention is easy to be manufactured and assembled and can provide preferable structural strength and comfortability.

### BRIEF DESCRIPTION OF THE DRAWINGS

FIG. 1 is a stereogram of the present invention;

FIG. 2 is a breakdown stereogram of the present invention;

FIG. 3 is a stereogram of a calf-supporting component of the present invention from another perspective;

FIG. 4 is a cross-sectional view of the present invention;

FIG. 5 is a partial breakdown stereogram of the present invention;

FIG. 6 is a cross-sectional view of the present invention from another perspective;

FIG. 7 is a stereogram of a second embodiment of the present invention;

FIG. 8 is a partial breakdown stereogram of the second embodiment of the present invention;

FIG. 9 is a cross-sectional view of the second embodiment of the present invention; and

FIG. 10 is a stereogram of a third embodiment of the present invention.

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

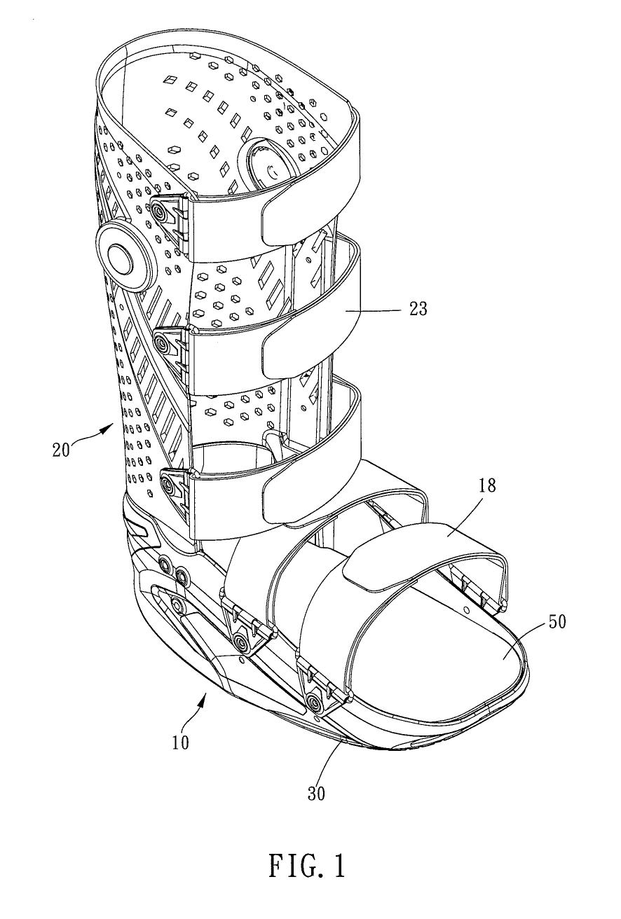

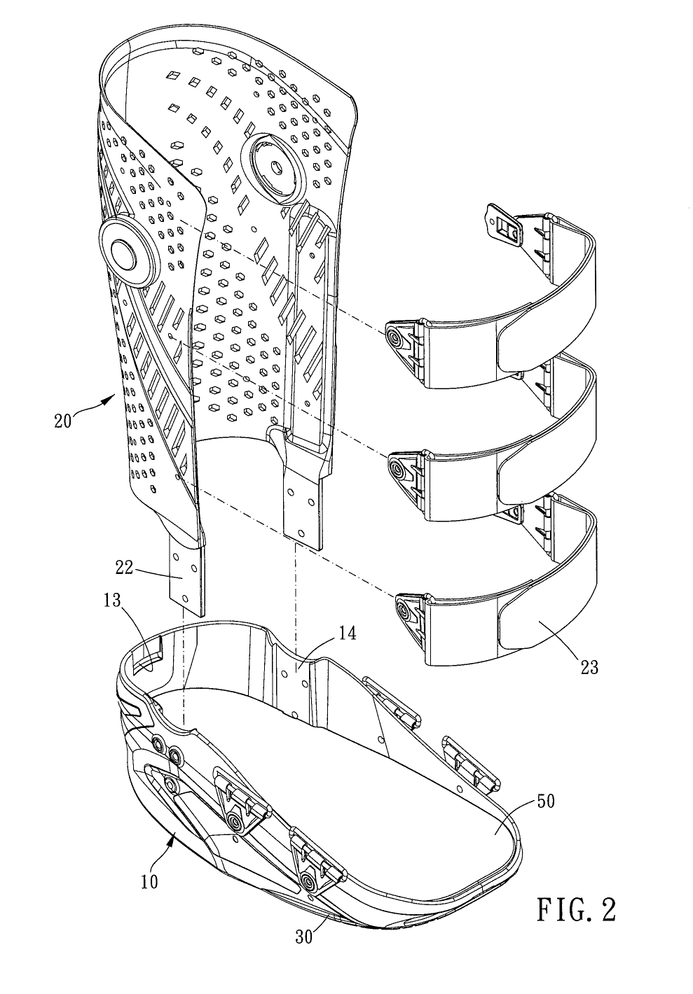

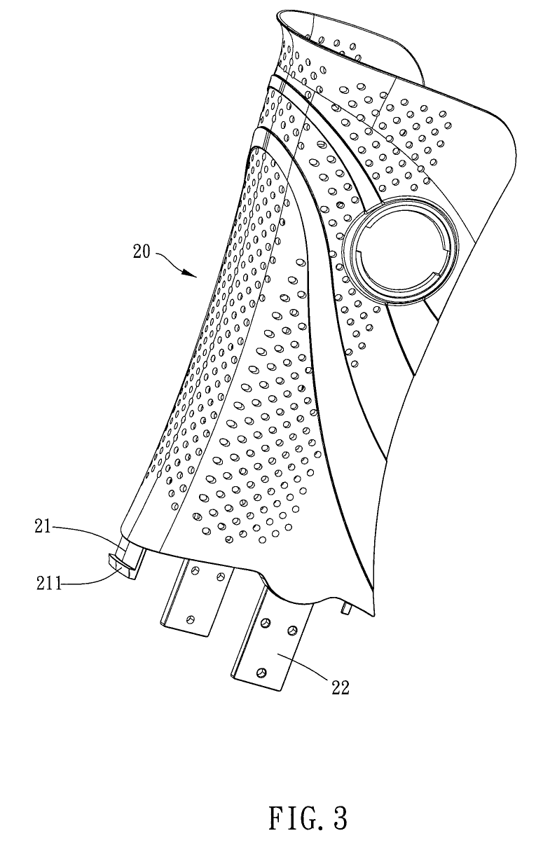

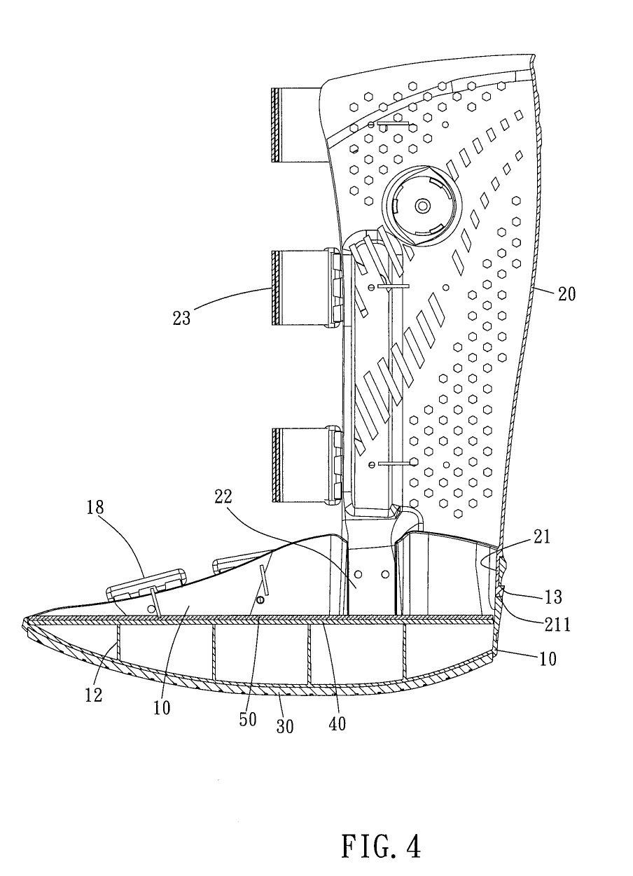

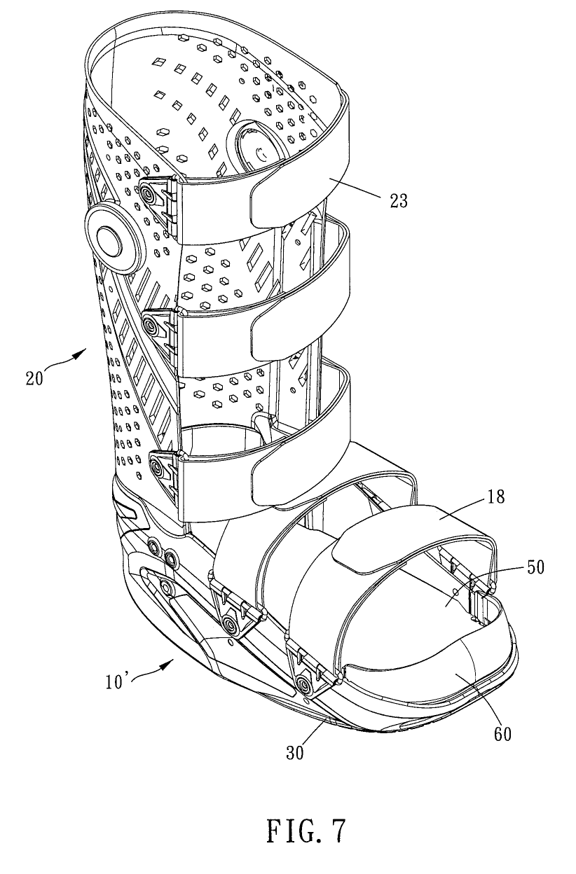

Please refer to FIGS. 1 to 10 for a rehabilitation shoe of the present invention, including a shoe shell 10, 10′, a calf-supporting component 20, a shoe sole 30, a pedal 40, 40′ and a shoe pad 50.

The shoe shell 10, 10′ has a plurality of first engaging portions 11 which extend horizontally and are near a bottom portion of the shoe shell 10, 10′; the calf-supporting component 20 is detachably arranged above a rear end of the shoe shell 10, 10′ and extends upward; the shoe sole 30 is made of a flexible material, an inner edge of a top portion of the shoe sole 30 has a plurality of second engaging portions 31 which extend horizontally, the second engaging portions 31 and the first engaged portions 11 are positionably engaged with each other so that the shoe sole 30 is fixed on the bottom portion of the shoe shell 10, 10′; the pedal 40, 40′ is made of a hard material and disposed on a top surface of the shoe shell 10, 10′; the shoe pad 50 is made of a soft material and disposed on a top surface of the pedal 40, 40′, and the shoe pad 50 is for a foot to step thereon.

Regarding the first engaging portion 11 and the second engaging portion 31, the first engaging portions 11 are protrusions which are spacingly arranged along an outer edge of the shoe shell 10, 10′, the second engaging portions 31 are recesses which are spacingly arranged along an inner edge of the shoe sole 30, and the first engaging portions 11 are at least distributed on right and left sides and a front end of the shoe shell 10, 10′. Because the shoe sole 30 is flexible, the first engaging portions 11 can be easily stuffed in the second engaging portions 31, and the shoe sole 30 can be engaged with the shoe shell 10, 10′ without additional fixing components.

Regarding an engagement of the shoe shell 10, 10′ and the calf-supporting components 20, the rear end of the shoe shell 10, 10′ has an engaging groove 13, the calf-supporting component 20 extends downward to form an extension 21, a lower end of the extension 21 has an engaging protrusion 211, and the engaging protrusion 211 is engaged within the engaging groove 13 so as to fix the calf-supporting component 20 and the shoe shell 10, 10′ together. In addition, inner edges of two sides of the rear end of the shoe shell 10, 10′ respectively form an inserted slot 14, two ends of the calf-supporting component 20 respectively extend downward to form an inserting block 22, and the two inserting blocks 22 are respectively inserted in the two inserted slots 14 and fixed to the shoe shell 10, 10′ (may also use positioning components like a inserting pin).

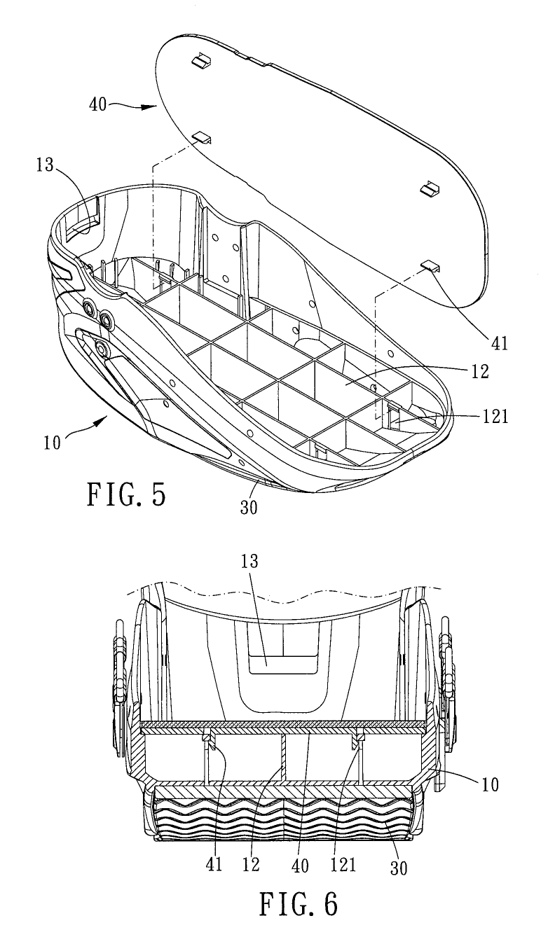

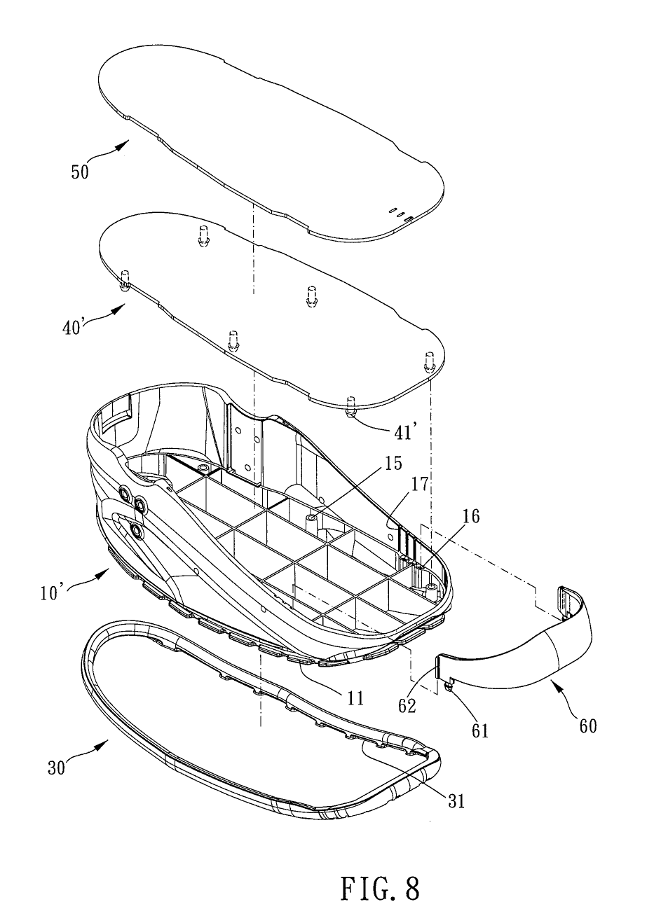

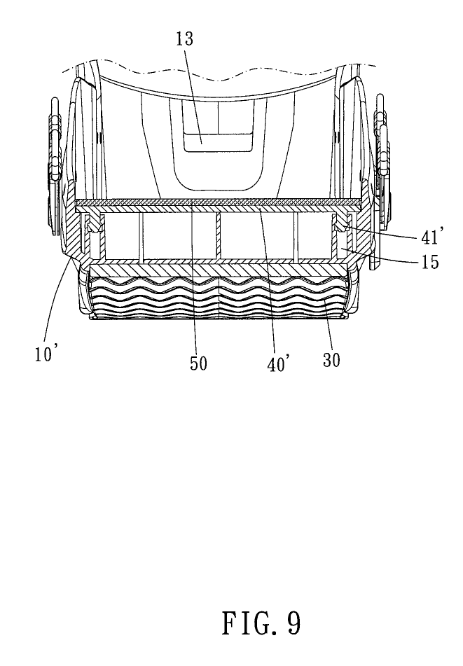

Regarding an engagement of the shoe shell 10, 10′ and the pedal 40, 40′, in a first embodiment, as shown in FIGS. 5 and 6, a top portion of the shoe shell 10, 10′ has a plurality of rib portions 12 which are vertical to the shoe shell 10, 10′, the rib portions 12 have a plurality of positioning holes 121, a bottom portion of the pedal 40 has a plurality of positioning blocks 41 which extend vertically downward, a bottom end of a side of each said positioning block 41 facing the positioning hole 121 protrudes and is engaged within the positioning hole 121 so as to make the pedal 40 positioned on the shoe shell 10. Specifically, a connection of the front end and the rear end of the shoe shell 10 defines a longitudinal direction and further defines a transverse direction, the transverse direction is perpendicular to a vertical direction and the longitudinal direction, the top portion of the shoe shell 10 has at least two said rib portions 12 which are arranged along the transverse direction and respectively extend along the longitudinal direction, and the positioning block 41 of the pedal 40 is located between the two rib portions 12. In a second embodiment, as shown in FIGS. 8 and 9, the top portion of the shoe shell 10′ has a plurality of positioning holes 15, the positioning holes 15 extend along the vertical direction, a bottom portion of the pedal 40′ has a plurality of positioning bolts 41′ which extend vertically downward, a distal end of each said positioning bolt 41′ has a radially-enlarged portion, the positioning bolts 41′ are for being inserted in the positioning holes 15, and the radially-enlarged portion positions the positioning bolt 41′ within the positioning hole 15.

Regarding the calf-supporting component 20 and the shoe shell 10, 10′, the calf-supporting component 20 is curved and complementary to a back side of a human calf in shape, at least one first retaining strap 23 is further provided, one of two ends of the first retaining strap 23 is fixed on one of two ends of the calf-supporting component 20, and the other of the two ends of the first retaining strap 23 is detachably positioned on the other of the two ends of the calf-supporting component 20. At least one second retaining strap 18 is further provided, the second retaining strap 18 is arranged above the shoe pad 50, one of two ends of the second retaining strap 18 is fixed on the top portion of one of two ends of the shoe shell 10, 10′, the other of the two ends of the second retaining strap 18 is detachably positioned on the top portion of the other of the two ends of the shoe shell 10, 10′.

In the second embodiment, a toe-stopping cover 60 is further provided, the toe-stopping cover 60 is curved and detachably disposed on the front end of the shoe shell 10′ so as to block the toes of the user and prevent the toes from sliding forward. Specifically, two ends of the toe-stopping cover 60 respectively extend downward to form a positioning bolt 61, a bottom portion of one of the two positioning bolts 61 has a radially-enlarged portion, outer sides of the two ends of the toe-stopping cover 60 respectively form a slide protrusion 62 which is elongated, two sides of the bottom portion of the front end of the shoe shell 10′ respectively farm at least one inserted hole 16, inner walls of two sides of the front end of the shoe shell 10′ respectively form at least one slide slot 17, the two positioning bolts 61 are respectively positionably inserted in the two inserted holes 16, the two slide protrusions 62 are respectively and slidably arranged within the two slide slots 17, and the inserted holes 16 are closer to the front end of the shoe shell 10′ than the slide slot 17. Preferably, the two sides of the bottom portion of the front end of the shoe shell 10′ respectively form the plurality of inserted holes 16, the inserting holes 16 are arranged along the longitudinal direction, the inner walls of the two sides of the front end of the shoe shell 10′ respectively form the plurality of slide slots 17, the slide slots 17 are arranged along the longitudinal direction, each said positioning bolt 61 is optionably inserted in one of the two inserted holes 16 so that a position of the toe-stopping cover can be adjusted according to a foot length of the user.

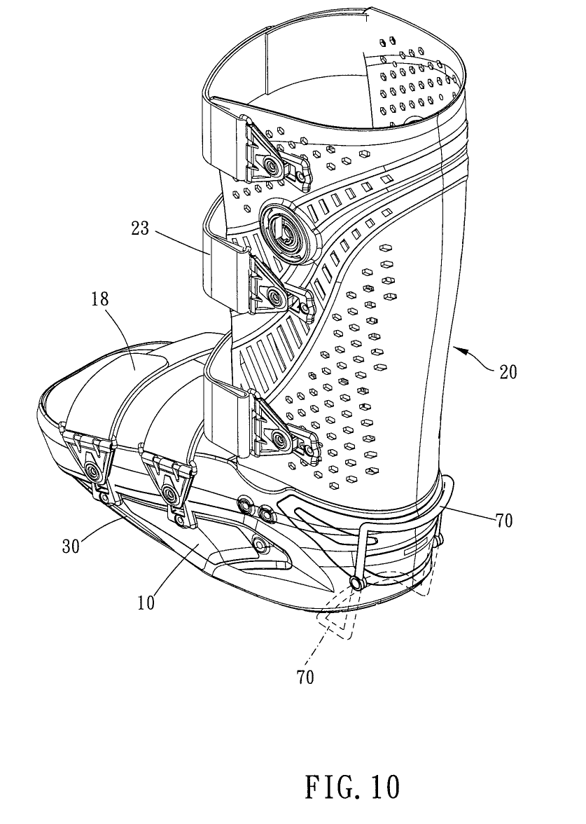

Please further refer to FIG. 10, the rehabilitation shoe further includes a supporting bracket 70, the supporting bracket 70 is substantially n-shaped, two ends of the supporting bracket 70 are respectively pivoted to two sides of the rear end of the shoe shell 10 to make the supporting bracket 70 swing between a storage position and a supporting position, when the supporting bracket 70 is on the storage position, the supporting bracket 70 is behind the shoe shell 10 and not lower than the bottom portion of the shoe shell 10, and when the supporting bracket 70 is on the supporting position, the supporting bracket 70 extends downward and is located below the shoe shell 10. When the user walks, the supporting bracket 70 can be swung to the storage position; and when the user is at rest, the supporting bracket 70 can be swung to the supporting position to enhance a supporting effect and prevent the user from falling.

Given the above, the rehabilitation shoe is composed simple components and can be easily manufactured and assembled. In addition, the rehabilitation shoe has preferable structural strength and shock absorption ability and provides great comfortability and supporting effect.

While we have shown and described various embodiments in accordance with the present invention, it should be clear to those skilled in the art that further embodiments may be made without departing from the scope of the present invention.

What is claimed is:

1. A rehabilitation shoe, including:
a shoe shell, having a plurality of first engaging portions which extend horizontally and are near a bottom portion of the shoe shell;
a calf-supporting component, detachably arranged above a rear end of the shoe shell and extending upward;
a shoe sole, made of a flexible material, an inner edge of a top portion of the shoe sole having a plurality of second engaging portions which extend horizontally, the second engaging portions and the first engaged portions being positionably engaged with each other so that the shoe sole is fixed on the bottom portion of the shoe shell;
a pedal, made of a hard material and disposed on a top surface of the shoe shell;
a shoe pad, made of a soft material and disposed on a top surface of the pedal, the shoe pad being for a foot to step thereon.

2. The rehabilitation shoe of claim 1, wherein a top portion of the shoe shell has a plurality of rib portions which are vertical to the shoe shell, the rib portions have a plurality of positioning holes, a bottom portion of the pedal has a plurality of positioning blocks which extend vertically downward, a bottom end of a side of each said positioning block facing the positioning hole protrudes and is engaged within the positioning hole so as to make the pedal positioned on the shoe shell.

3. The rehabilitation shoe of claim 2, wherein a connection of a front end and the rear end of the shoe shell defines a longitudinal direction and further defines a transverse direction, the transverse direction is perpendicular to a vertical direction and the longitudinal direction, the top portion of the shoe shell has at least two said rib portions which are arranged along the transverse direction and respectively extend along the longitudinal direction, and the positioning block of the pedal is located between the two rib portions.

4. The rehabilitation shoe of claim 1, wherein a top portion of the shoe shell has a plurality of positioning holes, the positioning holes extend along a vertical direction, a bottom portion of the pedal has a plurality of positioning bolts which extend vertically downward, a distal end of each said positioning bolt has a radially-enlarged portion, the positioning bolts are for being inserted in the positioning holes, and the radially-enlarged portion positions the positioning bolt within the positioning hole.

5. The rehabilitation shoe of claim 1, wherein the first engaging portions are protrusions which are spacingly arranged along an outer edge of the shoe shell, the second engaging portions are recesses which are spacingly arranged along an inner edge of the shoe sole, and the first engaging portions are at least distributed on right and left sides and a front end of the shoe shell.

6. The rehabilitation shoe of claim 1, wherein the rear end of the shoe shell has an engaging groove, the calf-supporting component extends downward to form an extension, a lower end of the extension has an engaging protrusion, and the engaging protrusion is engaged within the engaging groove so as to fix the calf-supporting component and the shoe shell together.

7. The rehabilitation shoe of claim 1, wherein inner edges of two sides of the rear end of the shoe shell respectively form an inserted slot, two ends of the calf-supporting component respectively extend downward to form an inserting block, and the two inserting blocks are respectively inserted in the two inserted slots and fixed to the shoe shell.

8. The rehabilitation shoe of claim 1, further including a toe-stopping cover, the toe-stopping cover being curved and detachably disposed on a front end of the shoe shell.

9. The rehabilitation shoe of claim 8, wherein two ends of the toe-stopping cover respectively extend downward to form a positioning bolt, a bottom portion of one of the two positioning bolts has a radially-enlarged portion, outer sides of the two ends of the toe-stopping cover respectively form a slide protrusion which is elongated, two sides of a bottom portion of the front end of the shoe shell respectively form at least one inserted hole, inner walls of two sides of the front end of the shoe shell respectively form at least one slide slot, the two positioning bolts are respectively positionably inserted in the two inserted holes, and the two slide protrusions are respectively and slidably arranged within the two slide slots.

10. The rehabilitation shoe of claim 9, wherein the connection of the front end and the rear end of the shoe shell defines the longitudinal direction, the two sides of the bottom portion of the front end of the shoe shell respectively form the plurality of inserted holes, the inserted holes are arranged along the longitudinal direction, the inner walls of the two sides of the front end of the shoe shell respectively form the plurality of slide slots, the slide slots are arranged along the longitudinal direction, and each said positioning bolt is optionably inserted in one said inserted holes.

11. The rehabilitation shoe of claim 9, wherein the inserted holes are closer to the front end of the shoe shell than the slide slot.

12. The rehabilitation shoe of claim 1, wherein the calf-supporting component is curved and complementary to a back side of a human calf in shape, at least one first retaining strap is further provided, one of two ends of the first retaining strap is fixed on one of two ends of the calf-supporting component, and the other of the two ends of the first retaining strap is detachably positioned on the other of the two ends of the calf-supporting component.

13. The rehabilitation shoe of claim 1, further including at least one second retaining strap, the second retaining strap arranged above the shoe pad, one of two ends of the second retaining strap being fixed on a top portion of one of two ends of the shoe shell, the other of the two ends of the second retaining strap being detachably positioned on the top portion of the other of the two ends of the shoe shell.

14. The rehabilitation shoe of claim 1, further including a supporting bracket, the supporting bracket being substantially n-shaped, two ends of the supporting bracket being respectively pivoted to two sides of the rear end of the shoe shell to make the supporting bracket swing between a storage position and a supporting position, when the supporting bracket is on the storage position, the supporting bracket is behind the shoe shell and not lower than a bottom portion of the shoe shell, and when the supporting bracket is on the supporting position, the supporting bracket extends downward and is located below the shoe shell.
